(19) United States
(12) Patent Application Publication
(10) Pub. No.: US 2010/0035993 A1
(43) Pub. Date: Feb. 11, 2010

(54) METHODS AND COMPOSITIONS COMPRISING 2-[(3-CHLOROPHENYL) AMINO] PHENYLACETIC ACID FOR HYPERPERMEABILITY AND NEOVASCULARIZATION DISORDERS OF THE RETINA

(76) Inventors: Margo P. Cohen, New York, NY (US); Clyde W. Shearman, West Chester, PA (US); Van-Yu Wu, Cherry Hill, NJ (US)

Correspondence Address: MORGAN, LEWIS & BOCKIUS LLP, 1701 MARKET STREET, PHILADELPHIA, PA, 19103-2921, US

(21) Appl. No.: 12/187,034

(22) Filed: Aug. 6, 2008

(51) Int. Cl. A61K 31/195; A61P 27/02

(52) U.S. Cl. 514/567

(57) ABSTRACT

Method of preventing or arresting the progression of hyperpermeability and neovascularization of the retina by administering to a patient at risk for retinal microvascular disease a composition comprising a compound or pharmaceutically acceptable salt thereof that lowers the ocular ratio of VEGF to PEDF.

### BACKGROUND OF THE INVENTION

The present invention is directed to compositions that ameliorate the ocular imbalance between angiogenic and anti-angiogenic factors that is casually implicated in the development of retinal hyperpermeability and neovascularization of the retina, as well as methods of use of these compositions for the prevention or arrest of progression of retinal microvascular disease. More particularly, it has been discovered that the anti-glycation compound 2-[(3-chlorophenyl)amino]phenylacetic acid (23CPPA) reduces the production by retinal cells of the angiogenic vascular endothelial growth factor (VEGF) and increases the production by retinal cells of the anti-angiogenic pigment endothelium derived factor (PEDF), and provides a method of preventing or arresting the development of retinal microvascular disease. The method includes the step of administering to a patient in need of such treatment a composition comprising the above compound or a pharmaceutically acceptable salt thereof in an amount sufficient to elicit a prophylactic or therapeutic effect.

This invention relates to the use of 2-[(3-chlorophenyl)amino]phenylacetic acid in the prevention and treatment of retinal microvascular disease. 23CPPA interacts with the binding pocket domains IIA and IIIA in the albumin molecule, rendering susceptible lysine amino groups in or near the binding pockets inaccessible for condensation with glucose in the reaction known as nonenzymatic glycation (U.S. Pat. Nos. 6,355,680 and 6,552,077). 23CPPA lowers the concentration of nonenzymatically glycated albumin, even in the presence of marked hyperglycemia, and lessens the pathophysiologic effects of glycated albumin in living organism (Cohen et al, Kid Int 61:2025-2032, 2002; 68:1554-1561, 2005; AJP Renal 292:789-795, 2007). 23CPPA does not have the molecular formula of a nonsteroidal anti-inflammatory (NSAID) drug, is not an isomer or enantioner of a NSAID, and is not a pharmacologic inhibitor of the cyclooxygenase enzymes COX1 and COX2.

Retinal microvascular disease, the most common cause of new cases of blindness in the United States, arises from injury to and loss of cells lining the smallest vessels nourishing the retina, dysregulation of capillary blood flow, compromise in the supply of oxygen to the cells of the retina and adjacent ocular compartments, and abnormal expression of the growth factors VEFG and PEDF that are elaborated by retinal cells. An imbalance in the production of the angiogenic and permeability-inducing VEGF and the anti-angiogenic and anti-vasopermeability serine protease inhibitor PEDF promotes the appearance and proliferation of renegade new vessels, resulting in neovascularization and leading to impairment of vision. Reduction in PEDF may initiate the overexpression of VEGF since PEDF downregulates VEGF expression; it also can exaggerate the deleterious effects of increased VEGF since PEDF reduces retinal vascular hyperpermeability and inhibits retinal angiogenesis (Stellmach et al, Proc Natl Acad Sci 98:2593-2597, 2001; Mori et al, J Cell Physiol 188:253-263, 2001; Gao et al, J Biol Chem 277:9492-9497, 2002; Liu et al, Proc Natl Acad Sci 101:6605-6610, 2004).

Retinal neovascularization occurs toward the vitreous compartment, with microproliferation and migration of cells onto the posterior vitreous cortex, giving rise to the vitreal presence of VEGF and PEDF (Aiello et al, N Engl J Med 331:1480-1487, 1994; Dawson et al, Science 285:245-258, 1999). The vitreous of patients with proliferative retinopathy, and the vitreous of diabetic rodents which have not yet manifested proliferative retinopathic changes, contain decreased levels of PEDF and increased levels of VEGF compared to the vitreous of nondiabetic counterparts (Gao et al, FEBS Lett 480:270-276, 2001; Ogata et al, Am J Ophthalmol 134:348-353, 2002; Duh et al, Am J Ophthalmol 137:668-674, 2004; Cohen et al, Ophthalmic Res 40:5-9, 2008). These reciprocal changes in the vitreous are exacerbated by vascular leakage from the retina or from the choriocapillaries towards the retina, with a self-reinforcing cycle of increased microvascular permeability resulting in part from increased expression of VEGF and VEGF receptors and decreased expression of PEDF. They also represent developing pathophysiology at an early stage, evidenced by the findings that vitreous of diabetic animals contains increased VEGF and decreased PEDF before abnormal proliferation of retinal capillaries becomes histopathologically demonstrated (Cohen et al, Ophthal Res 40:5-9, 2008), that VEGF is increased in the vitreous and in nonvascular cells in eyes from diabetic patients even without overt retinopathy (Amin et al, Invest Ophthal Vis Sci 38:36-47, 1997, Lutty et al, Arch Ophthalmol 114:971-977, 1996), and that increased VEGF and decreased PEDF also have been found in aqueous humor from diabetic patients (Boehm et al, Horm Met Res 35:382-386, 2003), even those with no or mild retinopathy (Boehm et al, Diabetologia 46:394-400, 2003). The present invention demonstrates that 23CPPA possesses the ability to raise PEDF and lower VEGF levels in the ocular fluid of experimentally diabetic rats by directly or indirectly modulating the processes underlying the imbalanced production of these growth factors that causally contributes to retinal microvascular disease.

### SUMMARY OF THE INVENTION

The present invention provides novel methods and compositions that attenuate the underproduction of PEDF and the overexpression of VEGF that are causally implicated in the development of retinal microvascular disease.

The present invention also provides novel methods and compositions for the prevention and arrest of progression of retinal microvascular disease. The method includes the step of administering to a patient in need of such treatment 23CPPA or pharmaceutically acceptable salt thereof in an amount sufficient to elicit a prophylactic or therapeutic affect.

In some embodiments, these and other objects of the invention are achieved with the discovery that the compound 23CPPA enhances the production of the anti-angiogenic, anti-vasopermeability PEDF and lowers the production of the angiogenic hyperpermeability-promoting VEGF and thereby may prevent or arrest the development of retinal microvascular disease.

### DETAILED DESCRIPTION OF THE INVENTION

It has been unexpectedly discovered as described in the present invention that 23CPPA increases the level of PEDF and decreases the level of VEGF in mammalian ocular fluid and therefore provides a method of preventing or arresting the development of retinal microvascular disease.

It is a novel and unanticipated finding of the present invention that the compound 23CPPA and its pharmaceutically acceptable salts possess the ability to modulate the imbalance of angiogenic and anti-angiogenic growth factors in ocular fluid that is causally implicated in retinal microvascular disease.

The compound(s) of the present invention modulate the abnormal generation of growth factors that give rise to the hyperpermeability and angiogenicity that underlie retinal microvascular disease. Since therapeutic concentrations of the compound(s) of the present invention are capable of increasing the formation of PEDF and reducing the formation of VEGF, the present inventions provides a novel method for the treatment of retinal microvascular disease.

This invention also provides therapeutic compositions comprising the above-described compound(s).

This invention further provides a method for preventing and treating retinal microvascular disease comprising administering to the patient an effective amount of a therapeutic composition comprised of the above-described compound(s) capable of modulating the abnormal expression of growth factors and a pharmaceutically acceptable carrier.

The present invention also comprises compounds as described above formulated into compositions together with one or more non-toxic physiologically acceptable carriers, adjuvants or vehicles which are collectively referred to herein as carriers, for parenteral injection, for oral administration in solid or liquid form, for topical administration, or the like. The compositions can be administered to humans either orally, parenterally (intravenously, intramuscularly, subcutaneously), intraocularly, or locally (powders, ointments or drops).

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and new aqueous carriers, diluents, solvents or vehicles include water, ethanol, polyols (propyleneglycol polyethyleneglycol, glycerol and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the active compound may be admixed with at least one inert customary, pharmaceutically acceptable carrier, excipients (or carrier) such as sodium citrate or dicalcium phosphate or (a) fillers or extenders, as for example, starches, lactose, sucrose, glucose, mannitol and silicic acid, (b) binders, as for example, carboxymethyl-cellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like. Solid dosage forms such as tablets, capsules, pills and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes.

The active compound(s) can be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents, commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring and perfuming agents. Suspensions, in addition to the active compounds, may contain suspending agents, as for example, ethoxylated isostearyl alcohols, polyethylene sorbitol and sorbitan esters, microcrystalline cellulose, aluminum metahydrozide, bentonite, agar-agar and tragacanth, or mixtures of the substances, and the like.

Dosage forms for topical administration of a compound of this invention include ointments, powders, sprays and inhalants. The active component is admixed under sterile conditions with a physiologically acceptable carrier and any preservative, buffers or propellants as may be required. Ophthalmic formulations, eye ointments, powders and solutions are also included as being within the scope of this invention.

Actual dosage levels of active ingredients in the compositions of the present invention may be varied so as to obtain an amount of active ingredient that is effective to obtain a desired therapeutic response for a particular composition and method of administration. The selected dosage level therefore depends upon the desired therapeutic effect, on the route of administration, on the desired duration of treatment and other factors.

The total daily dose of the compound(s) of this invention administered to a host in single or divided dose may be in amounts, for example, of 50 to about 1500 mg. Dosage unit compositions may contain such amounts or such submultiples therefore as may be used to make up the daily dose. It will be understood, however, that the specific dose level for any particular patient will depend on a variety of factors including body weight, general health, gender, diet, time and route of administration, rates of absorption and excretion, combination with other drugs, and the severity of the disease being treated. The dosage level may also depend on patient response as determined by measurement of one or more appropriate markers in suitable biological fluid or tissue at suitable intervals after administration.

The above disclosure generally describes the present invention. A more complete understanding can be obtained by reference to the following specific examples which are provided herein for purposes of illustration only, and are not intended to limit the scope of the invention. It should be appreciated by those of skill in the art, in light of the present disclosure, that many changes can be made in the specific embodiments which are disclosed and still obtain a like or similar results without departing from the spirit and scope of the invention.

The following examples are included to demonstrate embodiments of the invention.

### EXAMPLE 1

23CPPA raises PEDF and lowers VEGF in ocular fluid.

Vitreous was harvested from male rats that were rendered diabetic by intravenous injection of streptozotocin and that were treated for 26 weeks with 23CPPA (15 mg/kg/day) administered by gavage, and from age and gender matched diabetic and nondiabetic control rats. PEDF and VEGF were measured by immunoassay. The relative ratio of VEGF to PEDF in diabetic control rats and diabetic rats treated with 23CPPA was compared to that in nondiabetic control rats which was assigned an arbitrary value of 1.0. The VEGF to PEDF relative ratio was significantly greater in diabetic compared to nondiabetic controls and was significantly lower in diabetic rats treated with 23CPPA compared to diabetic control rats.

| | | | Relative Ratio |
|---|---|---|---|
| Experimental Group | VEGF (pg/ml) | PEDF (ng/ml) | VEGF:PEDF |
| Normal Control | 75 | 15.3 | 1.0 |
| Diabetic Control | 167 | 4.6 | 7.3 |
| Diabetic-23CPPA | 105 | 8.7 | 3.7 |

What is claimed is:

1. A method of preventing or arresting the progression of hyperpermeability and neovascularization of the retina by administering to a patient at risk for retinal microvascular disease a composition comprising a compound or pharmaceutically acceptable salt thereof that lowers the ocular ratio of VEGF to PEDF.

2. A method of preventing or arresting the progression of hyperpermeability and neovascularization of the retina by administering to a patient at risk for retinal microvascular disease a composition comprising an amount of 2-[(3-chlorophenyl)amino]phenylacetic acid or pharmaceutically acceptable salt thereof sufficient to elicit a prophylactic or therapeutic effect.

3. A method according to claim 1 wherein said composition is administered orally, parenterally, intraocularly or topically.

4. A method according to claim 2 wherein said composition is administered orally, parenterally, intraocularly or topically.

5. A method according to claim 1 wherein said composition comprises a pharmaceutically acceptable carrier.

6. A method according to claim 2 wherein said composition comprises a pharmaceutically acceptable carrier.

7. A method according to claim 5 wherein said composition is administered orally, parenterally, intraocularly or topically.

8. A method according to claim 6 wherein said composition is administered orally, parenterally, intraocularly or topically.
